(19) United States
(12) Patent Application Publication
(10) Pub. No.: US 2024/0226234 A1
(43) Pub. Date: Jul. 11, 2024

(54) WNT5A PEPTIDES FOR THE TREATMENT OF ACUTE MYELOGENOUS LEUKEMIA

(71) Applicant: WNTRESEARCH AB, Malmö (SE)

(72) Inventors: Sara Teresinha SAAD, Campinas (BR); Fernanda Marconi ROVERSI, Campinas (BR); Maura Lima Pereira BUENO, Campinas (BR)

(21) Appl. No.: 18/562,783

(22) PCT Filed: May 3, 2022
(86) PCT No.: PCT/EP2022/061843

§ 371 Date: Nov. 20, 2023

(30) Foreign Application Priority Data
Jun. 1, 2021 (EP) 21177228.0
Nov. 19, 2021 (EP) 21209291.0

(51) Int. Cl. A61K 38/17; A61K 45/06; A61P 35/02

(52) U.S. Cl. CPC A61K 38/1709; A61K 45/06; A61P 35/02

(57) ABSTRACT

A WNT5A peptide or derivatives thereof for use in treatment of acute myelogenous leukemia in a subject in need thereof. Further, the WNT5A peptide or derivatives thereof may be in combination with at least one chemotherapeutic drug or blood transfusion for use in treatment of acute myelogenous leukemia in a subject in need thereof.

### TECHNICAL FIELD

A WNT5A peptide or derivatives thereof for use in treatment of Acute myelogenous leukemia in a subject.

### BACKGROUND ART

Acute myelogenous leukemia (AML) is a cancer of the blood and bone marrow. AML is a disorder characterized by a clonal proliferation derived from primitive hematopoietic stem cells (HPCs) or progenitor cells. Abnormal differentiation of myeloid cells results in a high level of immature malignant cells and fewer differentiated red blood cells, platelets, and white blood cells. Occasionally, AML cells can form a solid tumor called a myeloid sarcoma or chloroma that can develop anywhere in the body. AML typically presents with a rapid onset of symptoms that are attributable to bone marrow failure and may be fatal within weeks or months when left untreated. The disease occurs at all ages.

Acute myelogenous leukemia is also known as acute myeloid leukemia, acute myeloblastic leukemia, acute granulocytic leukemia and acute nonlymphocytic leukemia.

The main treatment for most types of AML is intensive, often multidrug, chemotherapy, sometimes along with a targeted therapy drug. This might be further supplemented by a stem cell transplant or blood transfusion. Patients undergoing treatment of AML often experience significant side effects and an important part of the work of treating AML is to treat the side effects that occur as part of the treatment. Thus, there remains a need for the development of less toxic therapies for treatment of AML in patients.

On this background it is an object of the present invention to provide improved therapies for use in treatment of acute myelogenous leukemia that entail fewer side effects and comprise administering a therapeutic agent that is better tolerated by the patients. It is further an object of the present invention to improve the treatment of patients suffering from recurrent acute myelogenous leukemia.

### SUMMARY OF THE INVENTION

Thus, according to a first aspect of the present invention, there is provided a WNT5A peptide or derivatives thereof for use in treatment of acute myelogenous leukemia in a subject in need thereof, the WNT5A peptide comprising X_{A}DGX_{B}EL (SEQ. ID. NO. 2), or a formylated derivative thereof, wherein X_{A} is methionine (M) or norleucine, X_{B} is cysteine (C) or alanine (A).

The skilled person knows how to diagnose acute myelogenous leukemia. The diagnosis of AML in a patient is typically made on the basis of an overall assessment of information based on morphological examination of blood or bone marrow as well as assessment of blood or bone marrow with flow cytometry.

The mitogen-activated protein kinase (MAPK), also referred to as extracellular signal kinase (ERK), signal transduction pathway and the phosphatidylinositol 3-kinase (PI3K)-protein kinase B (AKT) signal transduction pathway both participate in multiple cellular events, such as cell survival, proliferation, differentiation, motility, and apoptosis, and have been implicated in the pathogenesis of leukemia. Abnormal activation of the PI3K and MAPK pathways has been reported in patients suffering from AML. The abnormal activation of the PI3K and MAPK pathways can be evaluated based on the phosphorylation levels of the proteins involved in the signaling pathways. Two such proteins are the ERK protein and the AKT protein which together with their phosphorylated proteins, p-ERK and p-AKT, may be measured quantitatively and semi-quantitatively using flow cytometry and western blotting, respectively. It is postulated that the effective targeting of components involved in the MAPK and PI3K signaling pathways could have an impact on the outcome of AML treatment, e.g. by the effective downregulation of the MAPK and PI3K signaling cascade.

According to one embodiment of the present invention, there is provided a WNT5A peptide or derivatives thereof for use in treatment of acute myelogenous leukemia in a subject in need thereof, wherein the WNT5A peptide derives from WNT5A protein according to SEQ ID NO: 1 and comprises amino acid sequence X_{A}DGX_{B}EL (SEQ. ID. NO. 2), or a formylated derivative thereof, wherein X_{A} is methionine (M) or norleucine, X_{B} is cysteine (C) or alanine (A), and wherein a blood sample and/or bone marrow sample obtained from said subject has an upregulated MAPK and/or PI3K pathway compared to a healthy subject.

In one embodiment, the blood sample and/or bone marrow sample obtained from said subject has a higher level of p-ERK and/or p-AKT compared to a healthy subject.

The inventors of the present invention have surprisingly found that the formylated hexapeptide fragment of the WNT5A protein, Foxy-5, is effective in reducing phosphorylation of the AKT and ERK proteins in AML cells, and thus that Foxy-5 downregulates the PI3K and MAPK signaling pathways in patients suffering from AML.

In the context of the present invention, the AML patient is a patient from which a blood sample and/or bone marrow sample display an upregulated MAPK and/or PI3K pathway compared to a healthy subject. The upregulated MAPK and/or PI3K pathway is a measure of an abnormal activation of the PI3K and MAPK signaling pathways. The abnormal activation can e.g. be observed as an increased phosphorylation, i.e. activation, of the proteins involved in the PI3K and MAPK pathways. The skilled person knows how to measure the phosphorylation of proteins involved in the PI3K and MAPK pathways, but two such methods are flow cytometry or semi-quantitatively with western blot.

AML cells are also known to produce high concentrations of reactive oxygen species (ROS) to sustain growth and migration (chemotaxis), thereby causing leukemic cells to spread. The inventors of the present invention have found that Foxy-5, i.e. a formylated hexapeptide fragment of the WNT5A protein effectively reduces the ROS production in AML cells of a patient diagnosed with AML.

Since a drug must be produced in large scale, the full length WNT5A protein presents several disadvantages as a drug candidate. Being a large protein, WNT5A protein requires a complex and lengthy synthesis just in order to produce the correct primary amino acid sequence and posttranslational modifications, and in addition WNT5A has a heparan sulphate-binding domain which may limit its distribution in the body. Therefore, a smaller peptide that mimics the effect of WNT5A is more preferred.

In one embodiment, the total length of the WNT5A peptide or derivatives thereof is equal to or less than 50 amino acids.

WNT5A peptides comprising the amino acid sequence XDGXEL also comprises WNT5A mimicking effects. The peptides of varying lengths and comprising the sequence XDGXEL (SEQ ID NO: 2) may be produced synthetically, for example by liquid phase or solid phase peptide synthesis. The peptide of varying lengths has 20 amino acids or less and more preferred 10 amino acids or less. Most preferably, the peptide is a hexapeptide consisting of 6 amino acids. In one embodiment the amino acid sequence is XDGXEL, wherein the X in position 1 is M or norleucine and X in position 4 is C or A. Advantageously, the WNT5A peptide according to the invention is a peptide selected from the group consisting of:

(SEQ. ID. NO. 3)
MDGCEL,
(SEQ. ID. NO. 4)
GMDGCEL,
(SEQ. ID. NO. 5)
EGMDGCEL,
(SEQ. ID. NO. 6)
SEGMDGCEL,
(SEQ. ID. NO. 7)
TSEGMDGCEL,
(SEQ. ID. NO. 8)
KTSEGMDGCEL,
(SEQ. ID. NO. 9)
NKTSEGMDGCEL,
(SEQ. ID. NO. 10)
CNKTSEGMDGCEL,
(SEQ. ID. NO. 11)
LCNKTSEGMDGCEL,
(SEQ. ID. NO. 12)
RLCNKTSEGMDGCEL,
(SEQ. ID. NO. 13)
GRLCNKTSEGMDGCEL,
(SEQ. ID. NO. 14)
QGRLCNKTSEGMDGCEL,
(SEQ. ID. NO. 15)
TQGRLCNKTSEGMDGCEL,
(SEQ. ID. NO. 16)
GTQGRLCNKTSEGMDGCEL,
and
(SEQ. ID. NO. 17)
LGTQGRLCNKTSEGMDGCEL.

In a preferred embodiment, the WNT5A peptide is MDGCEL (SEQ. ID. NO. 3). In a more preferred embodiment methionine in position 1 is derivatized as formylated methionine (N-formyl methionine). This formylated hexapeptide is denoted Foxy-5. The modification/derivatization (formylation) of one amino acid improves the effect of the peptide and makes it more effective and resistant to degradation in vivo.

In yet another embodiment it is contemplated that the peptide according to the invention is for use in treatment of acute myelogenous leukemia in a subject in need thereof comprising the following step: immediately after diagnosis of acute myelogenous leukemia, administer an effective amount of the peptide, optionally wherein said step is repeated at least 3 times a week for 2 weeks or more. Alternatively, in another embodiment, the peptide according to the invention is for use in treatment of acute myelogenous leukemia in a subject in need thereof comprising the following step: immediately after diagnosis of acute myelogenous leukemia, administer an effective amount of the peptide, optionally wherein said step is repeated daily for 2 weeks or more. The period for treatment of the acute myelogenous leukemia with said peptide may be for 4, 6, 8, 12 weeks or up to 1, 2 or 3 months or more.

Said peptide may also be administered during the treatment period with a chemotherapeutic drug. Foxy-5 does not impair the cytotoxic effect of chemotherapy. Chemotherapeutic drugs are generally very toxic and in addition to targeting tumour cells also destroy healthy cells. Due to the toxicity the chemotherapeutic drug may not be administered long enough or in a sufficiently high dosage to destroy or eliminate all cancer cells or hematopoietic stem cells since the adverse effect towards normal cells would outweigh any positive effects.

In one embodiment, the peptide according to the invention in combination with at least one chemotherapeutic drug or blood transfusion may be administered to a subject, wherein a blood sample and/or bone marrow sample obtained from said subject has an upregulated MAPK and/or PI3K pathway compared to a healthy subject. In another embodiment, the peptide according to the invention in combination with at least one chemotherapeutic drug or blood transfusion may be administered to a subject, wherein a blood sample and/or bone marrow sample obtained from said subject has a higher level of p-ERK and/or p-AKT compared to a healthy subject.

The advantage of administering said peptide after treatment with a chemotherapeutic drug is that the peptide is non-toxic to the healthy cells whilst it eliminates or reduces the number of hematopoietic stem cells that remain following treatment with the chemotherapy, to which hematopoietic stem cells are resistant.

It is also contemplated that the administration of a chemotherapeutic drug and the peptide according to the invention can be simultaneous or the peptide may be administered before, during and after bone marrow transplant until the chemotherapy is started or subsequent to the conclusion of the chemotherapeutic treatment.

Thus, such combinatorial treatment can result in an improved cancer treatment without increasing the side effects.

Administration in combination may be beneficial to the patient compliance as the time period in which treatment is on-going may be shortened.

Sequential administration, i.e. administering the peptide before or following the chemotherapeutic drug may be more efficient from an economic perspective since the peptide is costly.

In one embodiment, the at least one chemotherapeutic drug is selected from cytarabine, daunorubicin, idarubicin, midostaurin, gemtuzumab ozogamicin, Gilteritinib and combinations thereof. In a preferred embodiment the chemotherapeutic drug is administered in combination with the formylated derivative of the WNT5A peptide according to SEQ. ID. NO. 3.

The choice of suitable chemotherapeutic drug will depend on the patient, diagnosis, disease progression.

### BRIEF DESCRIPTION OF THE FIGURES

The invention will be described in more detail below by means of non-limiting examples of embodiments and with reference to the figures, in which:

FIG. 1 shows WNT5A gene expression in mononuclear cells isolated from bone marrow of Acute Myeloid Leukemia patients. WNT5A gene expression is diminished in mononuclear cells isolated from bone marrow of AML patients who have adverse cytogenetic risk when compared to expression in AML patients who have favorable and intermediate cytogenetic risk. The gene expression analysis was done using the Ohsu database (Nature, 2018) and included 371 patients with AML, at diagnosis (Gao et al., 2013; Cerami et al., 2012). Statistical analysis: Anova; *P<0.05; **P<0.01; ***P<0.001.

FIG. 2 shows that Foxy-5 treatment reduces the reactive oxygen species (ROS) production of leukemic cells. Production of ROS was determined after treatment of (A) U937 cells, (B) HL-60 cells, (C) KG1a cells, and (D) K562 cells. Leukemia cells were treated with Foxy-5 (50 μM and 100 μM) or with vehicle during 72 hours. Then, ROS production was detected by flow cytometry using staining with DFCHA (2′,7′-dichlorofluorescin diacetate) (mean of fluorescence intensity—MFI). Treatment with Foxy-5 reduced the production of ROS in leukemic cells in monoculture. Results are shown as mean±standard deviation of duplicate replicate and are representative of, at least, 3 independent experiments. Statistical analysis: t Student; *P<0.05; **P<0.01; ***P<0.001.

FIG. 3 shows the effect of Foxy-5 treatment on reducing leukemic cell growth. (A) U937, (B) HL60, (C) KG1a, and (D) K562 cells were treated with Foxy-5 (100 μM) or vehicle during 72 hours. Then, cell number was evaluated by manual counting of cells in monoculture or by Image J software analysis of 2D culture (presence of HS-5 mesenchymal stromal cells). The treatment with Foxy-5 reduced leukemia cell growth when compared to vehicle treated cells. Results are shown as fold change and are representative of, at least, 3 independent experiments. Statistical analysis: t Student; *P<0.05; **P<0.01; ***P<0.001.

FIG. 4 shows that the treatment of leukemic cells with Foxy-5 affects cell cycle progression. (A) U937, (B) HL60, (C) KG1a, and (D) K562 cells were treated with Foxy-5 (50 μM and 100 μM) or vehicle during 72 hours. Afterwards, cell cycle phases were evaluated by flow cytometry using RNAse and propidium iodide buffer. Foxy-5 treatment increased the leukemic cells in the G0/G1 cell cycle phase, mainly in the 2D culture system, when compared with vehicle treated cells. Results are shown as percentage and are representative of, at least, 3 independent experiments. Statistical analysis: t Student; *P<0.05; **P<0.01; ***P<0.001.

FIG. 5 shows protein expression analysis (Western blot) after treatment with Foxy-5. (A) U937 and (B) K562 leukemic cells were treated with Foxy-5 (50 μM or 100 μM) or with vehicle during 72 hours. Then, total extracts were obtained, and Western blot analysis were performed. Foxy-5 treatment reduced phosphorylation of AKT and ERK1/2 levels, suggesting a downregulation in survival-associated PI3K and MAPK pathways. Results are shown as mean±standard deviation of duplicate replicate and are representative of, at least, 3 independent experiments.

FIG. 6 shows that Foxy-5 treatment decreases cell chemotaxis and reduces actin polymerization of leukemic cells. Chemotaxis assay in (A) U937, (B) HL-60, (C) THP-1 and (D) K562 cells treated with Foxy-5 (100 μM) or vehicle. Leukemia cell lines were treated with Foxy-5 (100 μM) or vehicle for 72 hours and allowed to migrate in Transwell chambers toward CXCL12 or fetal bovine serum-containing media (positive control) or bovine albumin-media (negative control). After 24 h, migrated cells on the lower chamber membrane were counted by flow cytometry. The Foxy-5 treatment was able to reduce leukemia cells' chemotaxis toward CXCL12.

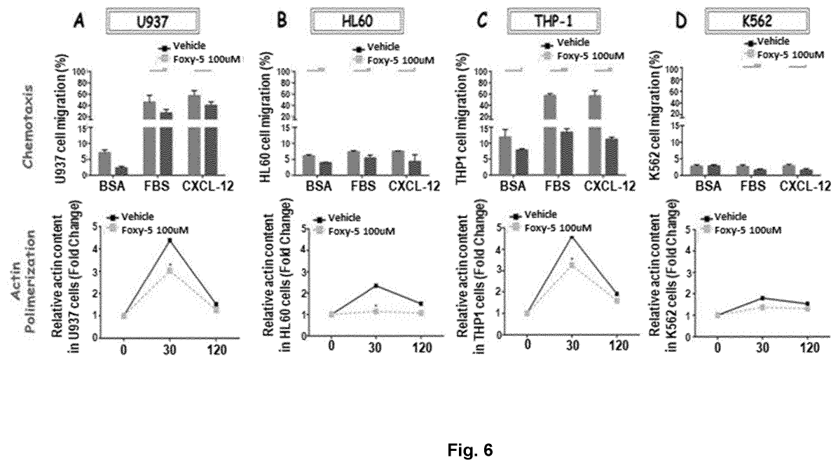

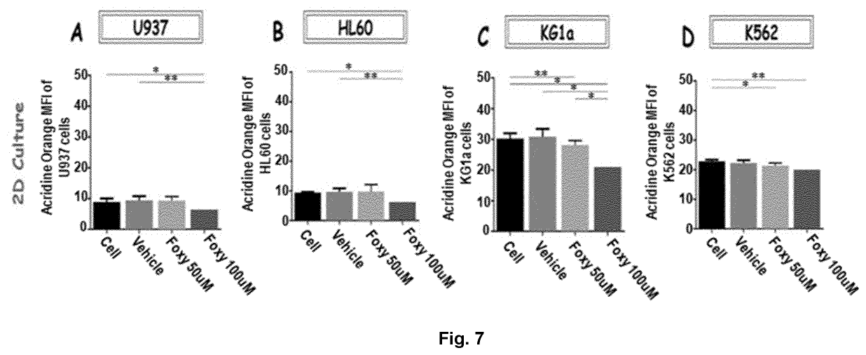

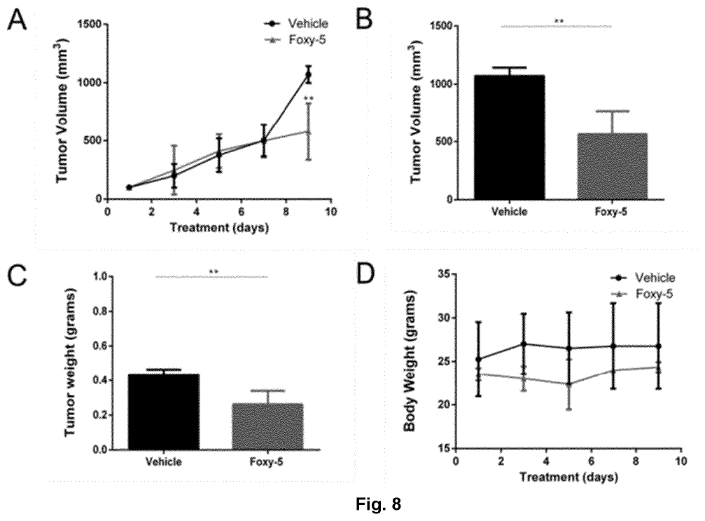

Actin polymerization assay in (A) U937, (B) HL60, (C) THP-1 and (D) K562 cells treated with Foxy-5 (100 μM) or vehicle is also shown in FIG. 6. Leukemia cell lines were treated with Foxy-5 (100 μM) or vehicle for 72 hours and stimulated with CXCL12 for 30 or 120 seconds. Intracellular F-actin content was measured by flow cytometry using phalloidin stain (mean of fluorescence intensity—MFI). Data indicate fold increase in F-actin content following stimulation with CXCL12. Results are shown as fold-change and are representative of, at least, 3 independent experiments. Statistical analysis: t Student; *P<0.05; **P<0.01; ***P<0.001.

FIG. 7 shows that the treatment with Foxy-5 reduces autophagy in leukemic cells. Leukemic (A) U937, (B) HL-60, (C) KG1a, and (D) K562 cells were treated with Foxy-5 (50 μM and 100 μM) or with vehicle during 72 hours. Then, autophagy was evaluated by acridine orange staining and detected by flow cytometry (mean of fluorescence intensity—MFI). Foxy-5 treatment reduced autophagy in leukemic cells in the 2D culture system. Results are shown as mean±standard deviation of duplicate replicate and are representative of, at least, 3 independent experiments. Statistical analysis: t Student; *P<0.05; **P<0.01; ***P<0.001.

FIG. 8 shows that the treatment with Foxy-5 reduces tumour growth in xenografts leukemic mice model. (A) tumour volume in mm³ of mice treated with Foxy-5 vs. vehicle; (B) excised tumour volume in mm³ of mice treated with Foxy-5 vs. vehicle; (C) tumour weight in grams (g) of mice treated with Foxy-5 vs. vehicle; and (D) body weight (grams, g) of the xenograft mice treated with Foxy-5 or vehicle. U937 cells were implanted subcutaneously into the flank of NOD/SCID mice. At least 3 mice were included in each group. NOD/SCID mice with U937 tumours were treated or not with Foxy-5 (40 μg/μL) once every 2 days during 9 days. Every 2 days, the tumour volumes were evaluated according to the following formula: tumour volume (mm³) ¼ (length×width²)/2. Results are shown as mean±standard deviation. Statistical analysis: paired Student T-Test; *P<0.05; **P<0.01; ***P<0.001.

### DETAILED DESCRIPTION

The Wnt (Wingless-related integration site) protein family contains highly conserved proteins that play a role in embryonic development such as body axis patterning, cell proliferation and migration. The Wnt signalling pathways are either canonical or non-canonical and they primarily trigger the regulation of gene transcription and increased proliferation via canonical signalling or regulation of several non-proliferative functions via activation of different non-canonical signalling pathways in the cells. The Wnt proteins are further involved in tissue regeneration in adult bone marrow, skin and intestine. Genetic mutation in the Wnt signalling pathway may cause breast cancer, prostate cancer glioblastoma, type II diabetes and other diseases.

The canonical Wnt pathway activates β-catenin and is integral in regulating self-renewal of normal stem cells and the subversion of the canonical Wnt signalling has been implicated in tumourigenesis. In contrast, non-canonical Wnt signalling is characterized by an absence of an increase in β-catenin signalling and has been studied for its role in embryonic patterning, gastrulation, and organogenesis. Moreover, non-canonical Wnt is proposed to antagonize canonical signalling. WNT5A is an example of a non-canonical Wnt ligand. WNT5A is tumour-suppressive in acute myelogenous leukemia (AML), colon cancer, breast and prostate cancer, and ovarian carcinoma. Over-expression of WNT5A in a WNT5A homozygous mouse model was shown to correlate with a reduced number of breast CSCs in a study by Borcherding et al., Paracrine WNT5A signalling inhibits expansion of tumour-initiating cells, Cancer Research 75:1972-1982, 2015 suggesting that heterozygous loss of WNT5A correlates with shorter survival of breast cancer patients. Interestingly, WNT5A has the opposite effect in malignant melanoma, gastric cancer as well as a few other cancer types, as exemplified by the fact that high expression of WNT5A in primary malignant melanoma is correlated with a shortened survival time.

WNT5A is a protein expressed by many normal cells in the body. WNT5A is secreted from the cells and exerts its action on the same or neighbouring cells by binding to and activating a receptor complex primarily involving a Frizzled receptor. The WNT5A protein is known to activate a receptor called Frizzled 5. Upon activation of the Frizzled 5 receptor a series of signalling events inside of the cells are activated, where one of the first events, is generation of short-lived increase in calcium inside of the cell, a so-called calcium-signal. The calcium-signal in turn triggers a series of forthcoming signalling events leading to a change in the functions of the cells, such as adhesion and migration. Thus, activating such a Frizzled receptor leads to signalling events inside the cell, resulting in increased adherence of the cell to its neighbouring cells and its adhesion to the surrounding connective tissue resulting in decreased ability of the tumour cell to migrate to structures in the vicinity, such as lymph nodes and blood vessels. In healthy breast epithelial cells for example, WNT5A is highly expressed and secures a firm adherence between cells and to the surrounding basement membrane and thereby restricts migration of the cells.

In order to reconstitute WNT5A signalling in cancer tissue that lack an endogenous expression of WNT5A, a small peptide, i.e. equal to or less than 20 amino acids derived from the amino acid sequence of the WNT5A molecule has been developed and then additionally modified. An example of such a peptide is Foxy-5, which is a true WNT5A agonist in that it triggers the same signaling events and functional responses as WNT5A and in comparison, with WNT5A it is much simpler molecule and it can be administered systemically and still reach the tumor. Thus, the term signalling properties, as used herein, means binding of the WNT5A or the Foxy-5 peptide to primarily a Frizzled receptor protein (Fz) followed by an intracellular signalling cascade in the cell eventually leading to modulation of AML cells.

The term modulation of AML cells, as used herein, is to be understood as affecting proliferation and/or migration of AML cells. In the context of the present inventive concept, proliferation is used interchangeably with cell growth.

The term blood transfusion, as used herein, is to be understood as stem cell and/or bone marrow transplantation. During the blood transfusion or stem cell transplantation, the blood stem cells will be replaced with new, healthy stem cells from a suitable donor.

AML cell lineages are known for producing high concentrations of ROS (Sillar et al., 2019), which enables the establishment of a hypoxic environment and activates critical pathways that induce growth and migration of leukemic cells, contributing to the progression of this neoplasm (Perillo et al., 2020). ROS production is considered tumorigenic through the ability to stimulate cell growth, survival, migration, and the induction DNA damage that sustain tumor progression (Liou & Storz, 2010). WNT5A-mimicking compound, Foxy-5 or a WNT5A peptide of the invention can modulating ROS production in patients diagnosed with leukemia and thus treatment of these patients with Foxy-5 causes a reduction in the ROS production. The lower rate of ROS production following Foxy-5 treatment or treatment with a WNT5A peptide of the invention is useful to prevent leukemogenesis.

Treatment with Foxy-5 compound reduce proliferation rates of leukemic cells. The cell growth is tightly correlated with the cell cycle and its regulation and treatment with Foxy-5 causes a G0/G1 cell cycle arrest. Cyclin D1 is an essential regulator of G1 to S phase transition. Foxy-5 or any WNT5A peptide of the invention reduces the protein level of cyclin D1 in leukemic cells treated with Foxy-5. The activity of PI3K and MAPK pathways are typically overexpressed in hematological malignancies and directly correlated with ROS production and cell growth activation (Braiacu et al., 2019; Sanches et al., 2019). Foxy-5 also reduce AKT and ERK protein phosphorylation levels after Foxy-5 treatment and thus downregulate the PI3K and MAPK pathways.

Foxy-5 or any WNT5A peptide of the invention can prevent CXCL12-induced chemotaxis of leukemia cells.

Foxy-5 or any WNT5A peptide of the invention can prevent the autophagy process, a process required for sustaining leukemia cell growth by induction of ROS production. In this way, treatment with Foxy-5 or any WNT5A peptide of the invention causes the reduction of autophagic events in the bone marrow niche and thus disrupt leukemia growth and maintenance.

The Foxy-5 or any WNT5A peptide of the invention is be an important modulating factor in biological processes associated with AML progression as this compound downregulates ROS production as well as cell growth, PI3K and MAPK pathways activation, chemotaxis and autophagy.

Foxy-5 or any WNT5A peptide of the invention reduces tumour growth in vivo in U937 xenograft mouse model. This further indicates the antiproliferative and antileukemia activity of Foxy-5 on AML patient cells. Thus, Foxy-5 or any WNT5A peptide of the invention is a promising therapeutic compound for treatment of Acute Myeloid Leukemia.

### Examples

To investigate WNT5A mRNA expression in AML patients and their impact in clinical outcomes and to analyze the effects of Foxy-5, a WNT5A-mimicking compound, in vitro (leukemia cell lines) and in vivo (xenograft mouse model), on Acute Myeloid Leukemia (AML) patients cells.

In the following, the methods used in the Examples 1 to 9 are presented first, followed by the results of the findings.

### Methods

### Reagents

Foxy-5 (WNT Research) was dissolved in NaHCO₃(sterile filtered 14 g/L) to a final concentration of 50 mM and then diluted in media for final concentration of 1 μM. Phorbol 12-myristate 13-acetate (PMA; Sigma) was diluted in DMSO to a 100 ng/μL stock solution. Recombinant Human SDF-1α (CXCL12, PeproTech) was diluted in saline buffer to a 100 ng/μL stock solution.

### Cell Culture

A panel of human myeloid leukemia cell lines (HL60, K562, KG1a, THP1 and U937) and HS-5 mesenchymal stromal cells lineage were cultured in Roswell Park Memorial Institute medium-1640 (RPMI; Sigma) containing 10% Fetal Bovine Serum (Gibco), glutamine (2 mM; Sigma), penicillin (100 μg/mL; Sigma), streptomycin (100 μg/mL; Sigma), and amphotericin B (0.25 μg/mL; Gibco). The cells were maintained in a humidified atmosphere at 37° C. and 5% CO2 and the experiments were performed when they reached exponential growth. For the assays, cells were seeded on a 24-well plate and treated or not with Foxy-5 for 72 hours.

### Reactive Oxygen Species (ROS) Production

Cells treated with Foxy or with vehicle were collected and intracellular ROS generation was measured by flow cytometry following staining with DCFDA (25 mmol/L; Sigma). Acquisition of cells was performed on a FACScalibur flow cytometer (BD) and analysis was carried out using the FlowJo software.

### Autophagy Assay

Cells treated with Foxy or with vehicle were collected and acids vesicles were measured by flow cytometry following staining with Acridine Orange (0.01 mg/mL; Sigma). Acquisition of cells was performed on a FACScalibur flow cytometer (BD) and analysis was carried out using the FlowJo software 4.

### Cell Growth Assay

Cells treated with Foxy or with vehicle were collected and cell number was evaluated by manual counting in monoculture and by microscopy with Image J software analysis in 2D culture.

### Cell Death/Apoptosis Assay

Cells treated with Foxy or with vehicle were tested and cell death was measured by Annexin-V and PI assay. Briefly, after cells were treated, they were washed and labeled with iodide propidium (1 μg/mL; Sigma) and APCAnnexin-V (1 μg/mL; BD). Ten thousand events were acquired for each sample and acquisition of cells was performed on a FACScalibur flow cytometer (BD) after incubation for 15 min at room temperature protected from the light. Analysis was carried out using the FlowJo software.

### Cell Differentiation Assay

Cells were subjected to treatment with Foxy-5 or with vehicle, in the presence or absence of PMA (200 nM). After 72 hours, adherent cells were washed with PBS and their morphology was examined using a Zeiss Axi-oskope 2 Plus microscope (Carl Zeiss AG). The program ImageJ was used to assess differentiation.

### Cell Cycle Assay

Cells treated with Foxy or with vehicle were collected and fixed, at least, 24 hours in 70% ethanol. DNA was stained with a buffer containing iodide propidium (20 μg/mL; Sigma) and RNase A (10 μg/mL; Sigma). Cell fluorescence was detected with a FACSCalibur flow cytometer (BD). The proportions of cells in the cell cycle phases were analyzed by Modifit (Verify Software House Inc), according to DNA distributions.

### Western Blotting

Equal amounts of protein were resolved by sodium dodecyl sulphate-polyacrylamide gel electrophoresis and transferred onto nitrocellulose membranes (Millipore). Detection was carried out with the chemiluminescent substrate using an ECL Plus (GE-Healthcare). Monoclonal antibodies against P70S6K (sc-8418) and polyclonal antibodies against Actin (sc-1616), AKT1/2/3 (sc-8312), BAX (sc-493), BCL-XL (sc-8392), GAPDH (sc-32,233), p-AKT1/2/3 (sc-33,437) and p-P70S6K (sc-7984) were purchased from Santa Cruz Biotechnology. ERK1/2 ([pT185/Y187]; 44-680G) and ERK1/2 (44-654G) from Invitrogen. Quantitative blots were carried out using the UN-SCAN-IT densitometer software.

### Chemotaxis Assay

Leukemic cell lines, pretreated or not with Foxy-5 (72 hours of stimulation) were tested for chemotaxis in Transwell-based assays for Chemotaxis (6.5 mm diameter, 8 μm pore size; Corning). Polycarbonate membranes were incubated with poly-L-lysine (1 mg/mL) for 1 hour at 37° C. followed by saline buffer washing. Briefly, cells were collected, washed with media containing bovine serum albumin (0.1% BSA; Sigma), and then seeded at a density of 5×105 cells into the upper chamber of the transwell. The lower chamber was filled with media containing 0.5% BSA (negative control), media containing 10% FBS (positive control) and media 5 containing 0.5% BSA with CXCL12 (100 ng/ml; Peprotech) (Melo et al., 2014). Following 6 h or 24 h of incubation at 37° C., cells that migrated into the lower chamber were collected, centrifuged, resuspended in saline buffer and counted. The number of migrated cells was expressed as a percentage of the input, i.e., the cells number applied directly to the lower compartment in parallel wells. The migration of cells was normalized to 100%+/−standard deviation (SD) of triplicates (Favaro et al., 2013).

### Actin Polymerization Assay

F-actin polymerization was analyzed in treated leukemic cell lines using AlexaFluor 633-labeled phalloidin (A22284, Invitrogen) before and after CXCL12 stimulation. The cells were stimulated with CXCL12 (300 ng/ml; Peprotech) in serum-free media at 37ºC for 30 and 120 seconds. The reaction was stopped by adding 3 volumes of paraformaldehyde (3.7%) at room temperature for 10 min, then washed and permeabilized on ice. Cells were stained with 633-phalloidin (2 mg/mL). Flow cytometric data were analyzed using a FACS flow cytometer (BD) followed by FlowJo software analysis. The F-actin content obtained were normalized calculating the ratio between each stimulated cells-mean fluorescence intensity (MFI) values and the nonstimulated cells MFI from the basal levels of the control cells and expressed as fold change.

### Xenograft Mouse Model

For the xenograft mouse model, a concentration of 40 μg/μL of Foxy-5 was used. 8-week-old female NOD/SCID (NOD.CB17Prkdcscid/JUnib) mice from The Jackson Laboratory, bred at the Animal Facility Centre at the University of Campinas, Brazil, under specific pathogen-free conditions, were matched for bodyweight before use. Mice were anesthetized with isoflurane (4%) and U937 cells (5,000,000 cells/mouse) were inoculated in the mouse dorsal region by subcutaneous injection. When the tumours reached 100 mm³, the treatment was initiated. Foxy-5 was administered once every 2 days by intraperitoneal injection. The control group received equal amounts of vehicle solution. The tumour volumes were measured every 2 days and evaluated according to the formula: tumour volume (mm³) ¼ (length×width²)/2. After 9 days, the mice were sacrificed, the tumour excised and tumour dimensions (tumour volume (mm³) and tumour weight (grams, g)) were evaluated.

### Statistical Analysis

Statistical analysis was performed using the GraphPad Prism5 software. Data were expressed as median [minimum-maximum]. For comparisons, Anova test or Student's t-test was used. The values of P<. 05 were considered as statistically significant. All experiments were repeated, at least, three independent times.

### Example 1: WNT5A Gene Expression in Acute Myeloid Leukemia Patients Mononuclear Cells

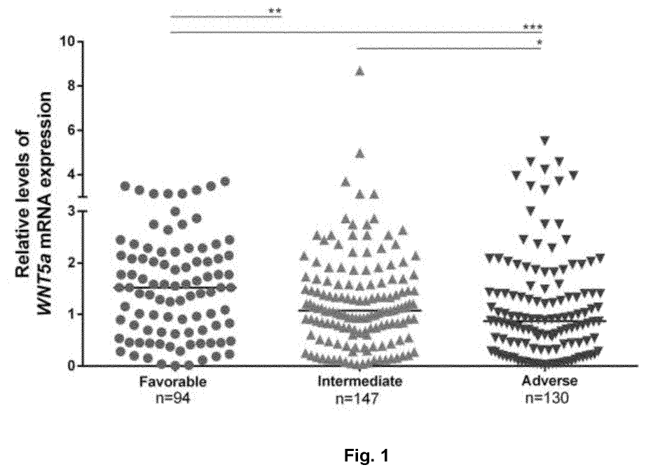

Initially, the WNT5A gene expression was evaluated in mononuclear cells isolated from the bone marrow of 371 patients with AML, at diagnosis, using the Ohsu database (Nature, 2018). We analyzed the gene expression in 94 samples of adverse cytogenetic risk AML patients, 94 samples of favorable cytogenetic risk AML patients and 147 samples of intermediate cytogenetic risk AML patients. The expression of WNT5A gene was reduced in bone marrow mononuclear cells isolated from patients with adverse cytogenetic risk (average: 1.144, range: 0.0093-5.5150) when compared with expression in AML patients with favorable (average: 1.1494, range: 0.0077-3.7010) and with intermediate cytogenetic risk (average: 1.262, range: 0.0319-8.6930) (P<0.05) (FIG. 1).

Using the Ohsu database (Nature, 2018) it was verified that WNT5A gene expression in mononuclear cells isolated from the bone marrow of AML patients with adverse cytogenetic risk was significant decrease when compared with AML patients with favorable and intermediate cytogenetic risk, probably indicating a correlation between WNT5A expression and the AML. prognosis.

### Example 2: The Biological Effects of Foxy-5, a WNT5A-mimicking Compound

In order to evaluate the role of WNT5A on leukemia pathophysiology and disease progression, we used Foxy-5, a WNT5A protein-mimicking compound synthesized by WNT Research. Foxy-5 effects were analyzed in different myeloid leukemia cell lines, U937, HL60, KG1a, THP-1 and K562, in monoculture and in 2D culture with the immortalized mesenchymal stromal lineage, HS-5. From the 5 myeloid leukemia cell lines, 4 were derived from acute myeloid leukemia (U937, KG1a/CD34+, HL60, and THP-1), and one from chronic myeloid leukemia/erythroleukemia (K562).

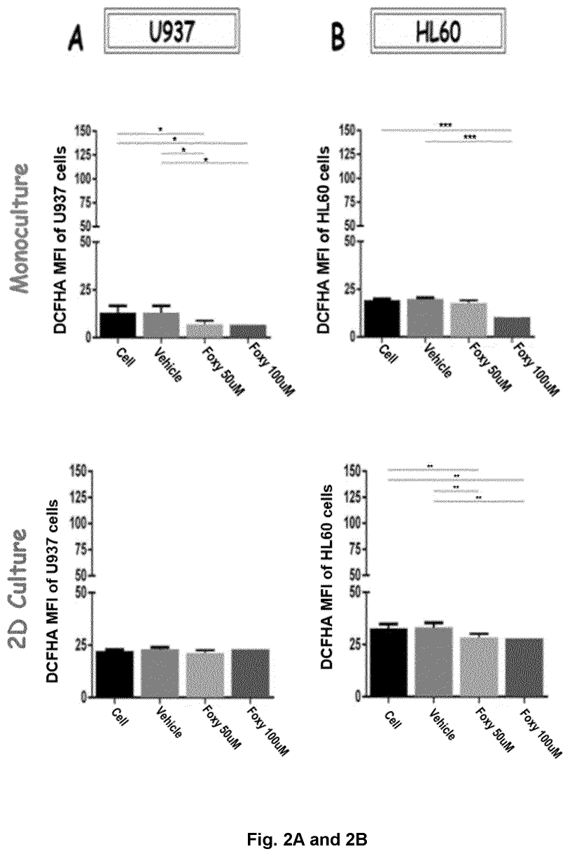

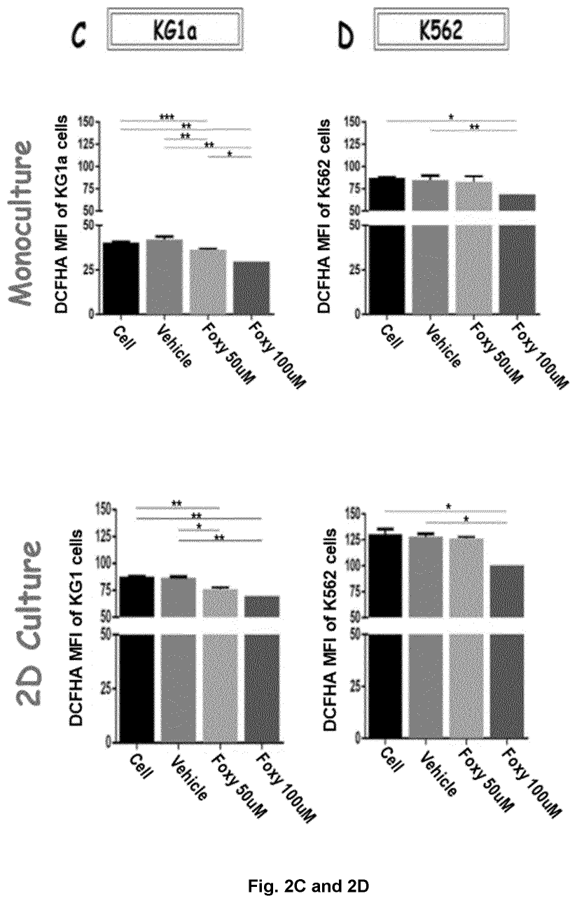

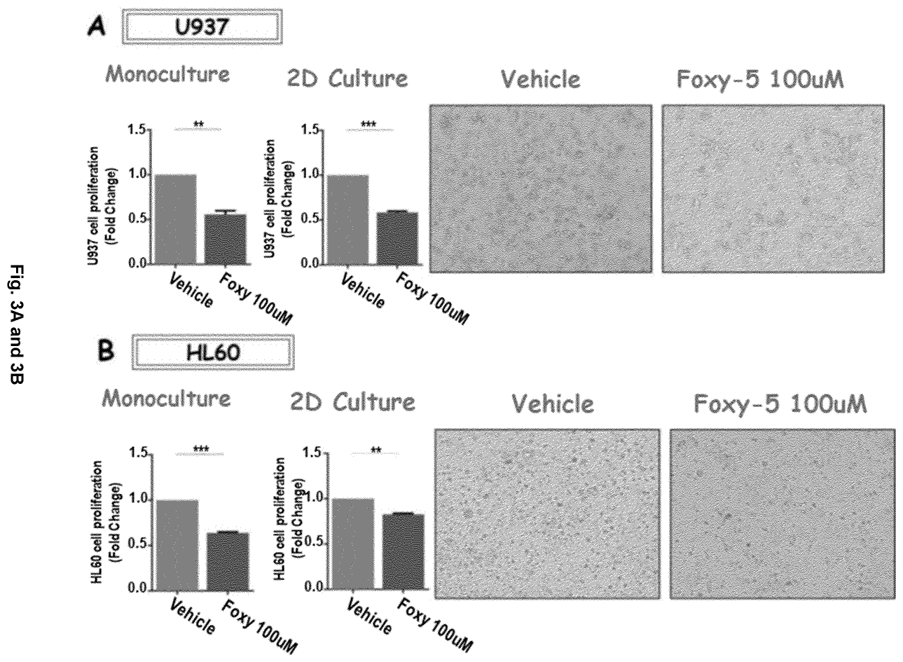

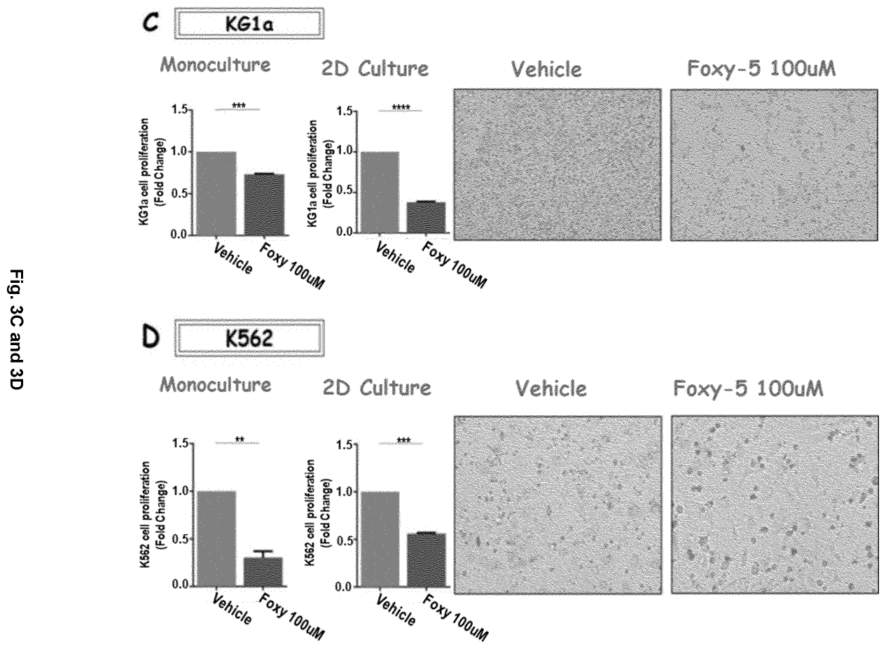

Since downregulation of Wnt5a protein mediates ROS generation that have been shown to be elevated in a wide range of cancers, including AML, we firstly investigated in a monoculture assay whether Foxy-5 is able to modulate ROS production in leukemia cells. Leukemia cell lines were treated for 72 h with Foxy-5, at doses of 50 and 100 μM, or vehicle, stained with DCFHA dye, and analyzed by flow cytometry. Results showed a significant reduction of ROS production (mean fluorescence intensity [MFI]): U937 cells-7.32 (range 4.80-8.67) and 7.09 (range 4.70-8.40) vs 13.70 (range 9.00-16.23), HL60 cells-17.45 (range 16.40-19.30) and 10.32 (range 10.10-10.80) vs 19.50 (range 19.24-20.74), KG1a cells-36.30 (range 35.80-36.80) and 29.72 (range 28.10-31.33) vs 42.03 (range 40.55-43.50), and K562 cells-83.90 (range 75.80-88.40) and 68.75 (range 64.40-72.75) vs 84.65 (range 79.50-89.80), respectively (P<0.05) (FIG. 2).

In addition, myeloid leukemia cells were seeded on a monolayer of mesenchymal stromal cells (HS5) and treated with Foxy-5 compound, at doses of 50 and 100 μM, or vehicle, for 72 hours. In these 2D-cultures, we also observed a significant decrease in ROS production (MFI): U937 cells-20.90 (range 19.90-22.63) and 23.50 (range 21.70-23.80) vs 23.20 (range 22.00-23.80), HL60 cells-28.52 (range 26.37-30.00) and 28.50 (range 25.03-30.30) vs 33.50 (range 30.97-35.20), KG1a cells-75.03 (range 75.03-77.70) and 69.00 (range 68.00-72.00) vs 87.00 (range 85.80-87.20), and K562 cells-126.00 (range 125.000-128.20) and 98.25 (range 98.25-104.00) vs 129.80 (range 125.00-129.80) respectively. (P<0.05) (FIG. 2).

Based on the monoculture and 2D culture assay, it was concluded that Foxy-5 is effective in reducing ROS production in leukemia cells.

### Example 3: Treatment with Foxy-5 Reduces Leukemic Cell Growth

Next, we evaluated the proliferation rates since this process is modulated by ROS. We treated the cells with Foxy-5 (100 μM) or with vehicle (DMSO) during 72 hours, and then cell growth was analyzed by counting the cells in monoculture, manually, or by the Image J software analysis of the 2D cultures. In the monoculture system, the treatment with Foxy-5 (100 μM) significantly reduced cell growth of U937 cells (1.75-fold decrease), HL60 cells (1.22-fold decrease), KG1a cells (1.37-fold decrease) and K562 cells (3.12-fold decrease) when compared to vehicle treated cells (P<0.001) (FIG. 3). Similar results were obtained in the 2D culture system using leukemia cells and HS-5 mesenchymal stromal cells. Foxy-5 significantly reduced cell growth of U937 cells (1.72-fold decrease), HL60 cells (1.56-fold decrease), KG1a cells (2.63-fold decrease) and K562 cells (1.79-fold decrease) when compared to vehicle treated cells (P<0.001) (FIG. 3).

Based on the monoculture and 2D culture assay, it was concluded that Foxy-5 is effective in reducing cell proliferation of leukemia cells.

### Example 4: Foxy-5 Treatment Induces an Arrest in G0/G1 Phase of Cell Cycle of Leukemic Cells

As we observed a reduction in the leukemic proliferation after Foxy-5 treatment, we also analyzed the cell cycle phases. Leukemia cell lines were treated for 72 hours with Foxy-5, at doses of 50 μM and 100 μM, or vehicle, stained with the Piper buffer containing RNAse and iodeto propidium and then analyzed by flow cytometry.

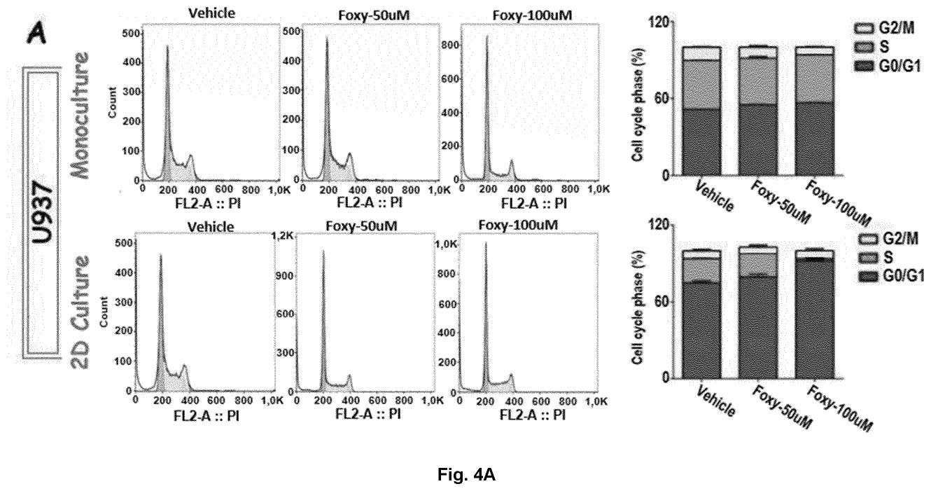

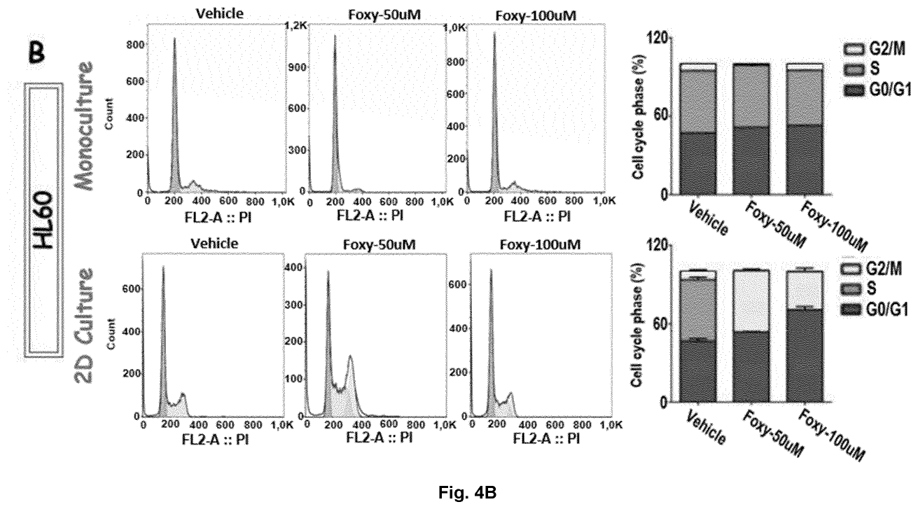

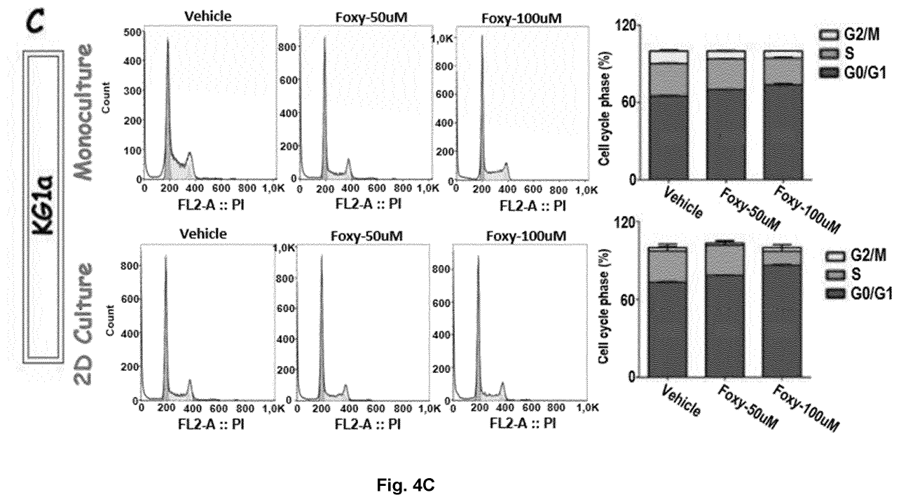

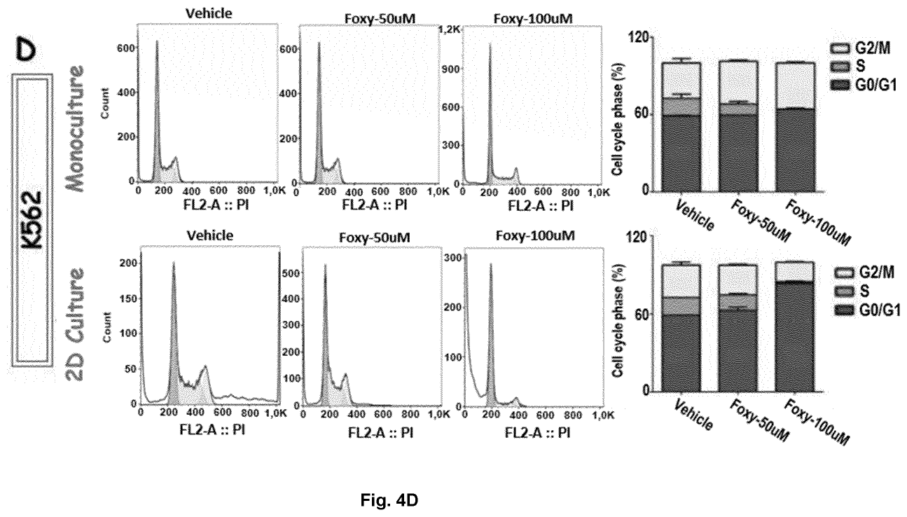

In monoculture, results revealed a significant G0/G1 cell cycle arrest in U937 cells-average 55.07% (range 54.52-55.62%) and average 56.64% (range 56.17-57.10%) vs. average 51.66%, (range 51.41-51.70%, HL60 cells-average 51.57% (range 51.56-51.58%) and average 53.02% (range 53.01-53.03%) vs. average 47.31% (range 47.30-47.32%), in KG1a cells-average 69.95% (range 69.69-70.20%) and average 73.79% (range 72.32-75.26%) vs. average 65.02% (range 64.19-65.85%), and K562 cells average 59.00% (range 59.00-59.62%) and average 83.51% (range 83.50 83.52%) vs. average 59.40% (range 58.81-59.50%) (P<0.001) (FIG. 4).

Similarly, when myeloid leukemia cells were co-cultured with HS5 mesenchymal stromal cells treated for 72 hours with Foxy-5, at doses of 50 and 100 μM, or vehicle, flow cytometry analyses also revealed a significant G0/G1 cell cycle arrest in co-cultured HS-5 cells, as U937 cells-average 77.37% (range 77.37-81.75%) and average 91.52% (range 90.93-93.92%) vs. average 74.88%, (range 72.23-77.52%), HL60 cells-average 53.47% (range 53.47-54.00%), and average 70.68% (range 66.33-75.03%) vs. average 47.31% (range 47.30-47.32%), KG1a cells-average 78.58% (range 78.23-79.37%) and average 76.53% (range 75.05-87.38%) vs. average 72.72%, (range 72.38-74.45%), and K562 cells-average 65.32% (range 60.30-63.32%) and average 83.52% (range 83.50-83.52%) vs. average 59.12% (range 59.12-59.18%) (P<0.001) (FIG. 4).

Thus, the leukemia cell treatment with Foxy-5 compound induces a G0/G1 cell arrest and prevents cells from entering the S phase, which may be associated with the decreased cell proliferation.

### Example 5: Foxy-5 Treatment Downregulates MAPK and PI3K Signaling Pathways

Based on the above results, the activity of proteins of PI3K and MAPK pathways were then investigated in a western blot. Briefly, U937 and K562 cells were seeded and treated with Foxy-5 (50 μM or 100 μM) or vehicle for 72 hours. Then, equal amounts of protein were used for total extracts followed by electrophoresis on 12% SDS polyacrylamide gels under reducing conditions, transferred to nitrocellulose membranes, blocked with 10% skimmed milk in TBS-0.1% Tween for 1 hour, stained with appropriate primary and secondary antibodies. The membranes were visualized with chemiluminescence using an ECL Plus (GE-Healthcare). Quantitative analyses of the optical intensities of the protein bands were determined with Un-Scan-It Gel 6.1 (Silk Scientific Inc.).

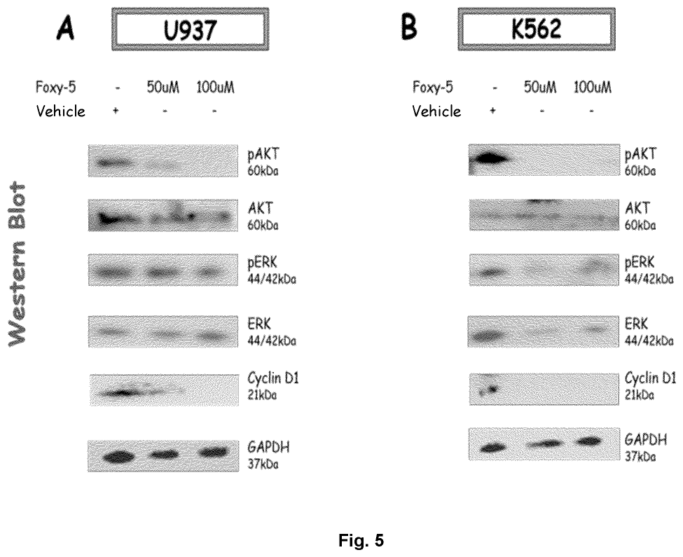

Protein expression analysis demonstrated lower phosphorylation levels of AKT and ERK in U937 and K562 cells treated with the Foxy-5 compared to vehicle-treated cells, thus suggesting a downregulation in survival-associated PI3K and MAPK pathway (FIG. 5).

In addition, a reduced protein level of cyclin D1 only was observed in U937 and K562 cells that were treated with Foxy-5 compared to vehicle-treated cells. The diminution of cyclin D1 protein levels, an essential regulator of the transition from G1 to S phase and, consequently, of cell cycle progression, corroborate the results of cell cycle arrest after Foxy-5 treatment (FIG. 5).

In conclusion, Foxy-5 is effective in downregulating the MAPK and PI3K signaling pathways.

### Example 6: Foxy-5 Reduces Chemotaxis Responsiveness to CXCL12 Stimulus and Actin Polymerization

We investigated whether restoring of WNT5A could enable the modulation CXCL12-induced chemotaxis of leukemia cells. Leukemic cells were pretreated with Foxy-5 (100 μM) or vehicle for 72 hours and then subjected to Transwell-based migration assays in the presence or absence of a stimulus, CXCL12 (100 ng/mL), for an additional 24 hours. Because KG1a cells do not express the SDF-1 receptor (CXCR-4), SDF-1 did not induce migration of KG1a cells, we performed migration assay with CD34-negative cells, but CXCR-4-positive leukemic cell line THP-1. Foxy-5 treatment resulted in reduction of CXCL12-induced chemotaxis compared to vehicle: U937 cells-average 40.35% (range 33.30-45.80%) vs. average 56.00% (range 48.70-67.80%); HL60 cells-average 4.31% (range 2.81-5.82%) vs. average 7.51% (range 7.45-7.57%); K562 cells-average 1.52% (range 1.10-2.04%) vs. average 2.77% (range 2.41-3.53%), and THP1 cells average 11.17% (range 10.68-11.65%) vs. average 56.67% (range 50.00-63.33%) (P<0.01) (FIG. 6).

Therefore, we performed a flow cytometry analysis to evaluate whether Foxy-5 treatment would be able to disrupt actin polymerization. Leukemia cell lines were treated with Foxy-5 (100 μM) or vehicle during 72 hours, stimulated with CXCL12 (300 ng/ml) for 30 or 120 seconds, and stained with anti-phalloidin antibody for detection of actin polymerization by flow cytometry. In all leukemia cell lines, CXCL12 stimulus (30 seconds) of vehicle-treated cells induced a more prominent conversion of globular into F-actin compared to Foxy-stimulated cells: U937 cells (4.38 and 3.03-fold increase), HL60 cells (2.34 and 1.14-fold increase), K562 cells (1.80 and 1.37-fold increase), and THP1 cells (4.60 and 3.56-fold increase). Notably, vehicle treated cells maintained the same amount of F-actin compared to Foxy-stimulated cell, even 120 seconds after CXCL12 stimulation: U937 cells (1.52 and 1.26-fold), HL60 cells (1.50 and 1.07-fold increase), K562 cells (1.53 and 1.31-fold increase) and THP1 cells (1.91 and 1.59-fold-fold) (FIG. 6). Thus, a significant reduction of actin polymerization was shown after Foxy-5 treatment when compared to untreated cells after 30 seconds of CXCL12 induction (P<. 001).

In conclusion, Foxy-5 is effective in reducing actin polymerization and chemotaxis in leukemia cells.

### Example 7: Treatment with Foxy-5 Reduces Autophagy in Leukemic Cells

As autophagy is required for sustaining leukemia cell growth by induction of ROS production, we measured the levels of acidic granule formation by flow cytometry after Foxy-5 or vehicle treatment. In 2D culture, the treatment during 72 hours with 50 μM and 100 μM of Foxy-5 compound or vehicle, respectively, reduced autophagy in U937 cells (average 9.40 (range 7.68-11.00) and average 6.30 (range 4.80-8.31) vs. average 9.84 (range 7.40-10.60)), HL60 cells (average 10.04 (range 7.20-11.93) and average 6.60 (range 5.42-6.69) vs. average 10.30 (range 8.32-10.40)), KG1a cells (average 27.30 (range 27.20-29.83) and average 21.55 (range 19.05-22.40) vs. average 29.40 (range 29.30-33.85)), and K562 cells (average 21.32 (range 20.46-22.28) and average 19.80 (range 19.70-20.40) vs. average 22.20 (range 21.30-23.20) (all P<0.05) (FIG. 7).

Thus, the reduction of autophagic events in the bone marrow niche after Foxy-5 treatment cause disruption of leukemia growth and maintenance.

### Example 8: Foxy-5 Treatment Impairs Cell Differentiation

PMA reagent is a typical inducer of AML cell differentiation through generation of ROS. As U937 leukemia cell lineage corresponds to a model of differentiated monocyte and/or macrophage following treatment with PMA, we examined the effects of Foxy-5 treatment on U937 differentiation-inducing PMA. Following treatment with PMA for 72 hours, cells were examined by microscopy to detect morphology. The majority of cells treated with vehicle and stimulated with PMA differentiated into macrophages and adhered to the plates. Treatment of U937 cells with Foxy-5 reduced PMA-mediated differentiation when compared with PMA-stimulated cells treated with vehicle.

It was concluded that U937 cells treated with Foxy-5 were less responsive to PMA-induced differentiation.

### Example 9: Foxy-5 Reduces Tumour Growth in U937 Xenograft Mouse Model

The aim of this study was to investigate the effects of Foxy-5, a WNT5A-mimicking compound, in vivo using a xenograft mouse model, on Acute Myeloid Leukemia (AML) patients cells.

Foxy-5 has been shown in vitro to reduce proliferation rates of leukemia cells, including U937 cell lines. To examine the role of Foxy in vivo, a xenograft mouse model was developed in which U937 leukemia cells were inoculated into the flank of a NOD/SCID mouse. After the engraftment and growth of the tumour, Foxy-5 was administered once every 2 days by intraperitoneal injection. Xenografted NOD/SCID mice treated with Foxy-5 displayed a significant decrease in the tumour volume (median: 469 mm³, range: 411-859 mm³) compared to the control group treated with a vehicle (median: 1053 mm³, range: 1006-1172 mm³), see FIG. 8. Treatment of mice with 40 μg/μL of Foxy-5 resulted in 55.46% inhibition of tumour growth, see FIGS. 8A and 8B, and 45.51% inhibition of tumour weight, see FIG. 8C, in U937 xenografts when compared to the control group at day 9. The body weights of the xenograft mice did not vary significantly between different treatment groups after the 9 days, see FIG. 8D.

In conclusion, based on the in vivo studies using a U937 xenograft mouse model, it has been shown that treatment with Foxy-5 results in a reduced tumour volume compared to a control group treated with vehicle, thus indicating an antiproliferative and antileukemia activity of Foxy-5 on AML patient cells. Thus, Foxy-5 is a promising therapeutic compound for treatment of acute myeloid leukemia.

### REFERENCES

Asem M S, Buechler S, Wates R B, Miller D L, Stack M S. Wnt5a Signaling in Cancer. Cancers. 2016; 8(79): 1-18. https://doi:10.3390/cancers8090079.
Bernasconi P, Borsani O. Targeting Leukemia Stem Cell-Niche Dynamics: A New Challenge in AML Treatment. J Oncol. 2019; 2019:8323592. doi: 10.1155/2019/8323592.
Braicu C B, Buse M, Busuioc C, Drula R, Gulei D, Raduly L, et al. (2019). A Comprehensive Review on MAPK: A Promising Therapeutic Target in Cancer. Cancers (Basel). 11(10): 1618.
Cerami E, Gao J, Dogrusoz U, Gross B E, Sumer S O, Aksoy B A, Jacobsen A, Byrne C J, Heuer M L, Larsson, Antipin Y, Reva B, Goldberg A P, Sander C, Schultz N. The cBio Cancer Genomics Portal: An Open Platform for Exploring Multidimensional Cancer Genomics Data. Cancer Discovery. 2012; 2(5), 401 L P-404. https://doi.org/10.1158/2159-8290.CD-12-0095.
Favaro P, Traina F, Machado-Neto J A, Lazarini M, Lopes M R, Pereira J K, et al. (2013). FMNL1 promotes proliferation and migration of leukemia cells. J Leukoc Biol. 94(3):503-12. doi: 10.1189/jlb.0113057.
Gao J, Aksoy B A, Dogrusoz U, Dresdner G, Gross B, Sumer S O, Sun Y, Jacobsen A, Sinha R, Larsson E, Cerami E, Sander C, Schultz N. Integrative analysis of complex cancer genomics and clinical profiles using the cBioPortal. Sci Signal. 2013; 6(269):pl1. doi: 10.1126/scisignal.2004088.
Giacinti C, Giordano A. RB and cell cycle progression. Oncogene. 2006; 25:5220-5227. doi: 10.1038/sj.onc. 1209615.
Kerekes K, Banyai, L, Patthy L. Wnts grasp the WIF domain of Wnt Inhibitory Factor 1 at two distinct binding sites. EBS Lett. 2015; 589(20 Pt B):3044-51. doi: 10.1016/j.febslet.2015.08.031.
Liou G Y, Storz P. Reactive oxygen species in cancer. Free Radic Res. 2010; 44(5):479-96. doi: 10.3109/10715761003667554.
Luis T C, Ichii M, Brugman M H, Kincade P, Staal F J. Wnt signaling strength regulates normal hematopoiesis and its deregulation is involved in leukemia development. Leukemia. 2012; 26(3):414-21. doi: 10.1038/leu.2011.387.
Melo R C C, Longhini A L, Bigarella C L, Baratti M O, Traina F, Favaro P, et al. (2014). CXCR7 Is Highly Expressed in Acute Lymphoblastic Leukemia and Potentiates CXCR4 Response to CXCL12. PLOS One. 9(1): e85926. doi: 10.1371/journal.pone.0085926.
Nusse R, Clevers H. Wnt/-Catenin Signaling, Disease, and Emerging Therapeutic Modalities. Cell 2017, 169, 985-999.https://doi.org/10.3390/cells10010142
Pavlaki K, Pontikoglou C G, Demetriadou A, Batsali A K, Damianaki A, Simantirakis E, Kontakis M, Galanopoulos A, Kotsianidis I, Kastrinaki M C, Papadaki H A. Impaired proliferative potential of bone marrow mesenchymal stromal cells in patients with myelodysplastic syndromes is associated with abnormal WNT signaling pathway. Stem Cells Dev. 2014; 23(14): 1568-81. doi: 10.1089/scd.2013.0283.
Perillo B, Di Donato M, Pezone A, Di Zazzo E, Giovannelli P, Galasso G, Castoria G, Migliaccio A. ROS in cancer therapy: the bright side of the moon. Exp Mol Med. 2020; 52(2): 192-203. doi: 10.1038/s12276-020-0384-2.
Sanchez V E, Nichols C, Kim H N, Gang E J, Kim Y M. (2019). Targeting PI3K Signaling in Acute Lymphoblastic Leukemia. Int J Mol Sci. 20(2): 412-426.
Shen Y L, Luo Q, Guo Y X, Zheng G H, Yu J, Xu Y H. Bone marrow mesenchymal stem cell-derived Wnt5a inhibits leukemia cell progression in vitro via activation of the non-canonical Wnt signaling pathway. Oncol Lett. 2014; 8(1):85-90. doi: 10.3892/ol.2014.2117.
Sillar J R, Germon Z P, Deluliis G N, Dun M D. The Role of Reactive Oxygen Species in Acute Myeloid Leukaemia. Int J Mol Sci. 2019; 20(23):6003. doi: 10.3390/ijms20236003.
Valencia A, Román-Gómez J, Cervera J, Such E, Barragán E, Bolufer P, Moscardó F, Sanz G F, Sanz M A. Wnt signaling pathway is epigenetically regulated by methylation of Wnt antagonists in acute myeloid leukemia. Leukemia. 2009; 23(9):1658-66. doi: 10.1038/leu.2009.86.
Zang S, Liu N, Wang H, Wald D N, Shao N, Zhang J, Ma D, Ji C, Tse W. Wnt signaling is involved in 6-benzylthioinosine-induced AML cell differentiation. BMC Cancer 2014; 14:886. doi: 10.1186/1471-2407-14-886.

1. A method of treating acute myelogenous leukemia, comprising administering to a subject in need thereof a WNT5A peptide that comprises amino acid sequence X_{A}DGX_{B}EL (SEQ. ID. NO. 2), or a formylated derivative thereof, wherein X_{A} is methionine (M) or norleucine, X_{B} is cysteine (C) or alanine (A).

2. The method of to claim 1, wherein a blood sample and/or bone marrow sample obtained from said subject has an upregulated MAPK and/or PI3K pathway compared to a healthy subject.

3. The method of claim 1, wherein a blood sample and/or bone marrow sample obtained from said subject has a higher level of p-ERK and/or p-AKT compared to a healthy subject.

4. The method of claim 1, wherein the total length of the peptide is equal to or less than 50 amino acids.

5. The method of claim 1, wherein the WNT5A peptide is selected from the group consisting of:
MDGCEL (SEQ. ID. NO. 3),
GMDGCEL (SEQ. ID. NO. 4),
EGMDGCEL (SEQ. ID. NO. 5),
SEGMDGCEL (SEQ. ID. NO. 6),
TSEGMDGCEL (SEQ. ID. NO. 7),
KTSEGMDGCEL (SEQ. ID. NO. 8),
NKTSEGMDGCEL (SEQ. ID. NO. 9),
CNKTSEGMDGCEL (SEQ. ID. NO. 10),
LCNKTSEGMDGCEL (SEQ. ID. NO. 11),
RLCNKTSEGMDGCEL (SEQ. ID. NO. 12),
GRLCNKTSEGMDGCEL (SEQ. ID. NO. 13),
QGRLCNKTSEGMDGCEL (SEQ. ID. NO. 14),
TQGRLCNKTSEGMDGCEL (SEQ. ID. NO. 15),
GTQGRLCNKTSEGMDGCEL (SEQ. ID. NO. 16), and
LGTQGRLCNKTSEGMDGCEL (SEQ. ID. NO. 17), or a formylated derivative thereof.

6. The method of claim 1, wherein the WNT5A peptide is MDGCEL (SEQ. ID. NO. 3), or a formylated derivative thereof.

7. The method of claim 1, wherein said WNT5A peptide or derivative thereof is administered to said subject comprising the following steps:
a) immediately after diagnosis of acute myelogenous leukemia, administering an effective amount of the WNT5A peptide or derivatives thereof, optionally wherein step a) is repeated at least 3 times a week for 2 weeks or more.

8. The method of claim 1, wherein said WNT5A peptide or derivative thereof is administered to said subject comprising the following steps:
a) immediately after diagnosis of acute myelogenous leukemia, administering an effective amount of the WNT5A peptide or derivatives thereof, wherein step a) is repeated daily for 2 weeks or more.

9. The method of claim 1 wherein the WNT5A peptide or derivatives thereof is administered in combination with at least one chemotherapeutic drug or blood transfusion, wherein said peptide and said tumour suppressing chemotherapeutic drug are either combined or separate and/or are administered either simultaneously or sequentially.

10. The method of claim 9, wherein a blood sample and/or bone marrow sample obtained from said subject has an upregulated MAPK and/or PI3K pathway compared to a healthy subject.

11. The method of claim 9, wherein a blood sample and/or bone marrow sample obtained from said subject has a higher level of p-ERK and/or p-AKT compared to a healthy subject.

12. The method of claim 9, wherein said at least one chemotherapeutic drug is selected from cytarabine, daunorubicin, idarubicin, midostaurin, gemtuzumab ozogamicin, Gilteritinib and combinations thereof.

13. The method of claim 9, wherein the total length of the peptide is equal to or less than 50 amino acids.

14. The method of claim 9, wherein said WNT5A peptide is MDGCEL (SEQ. ID. NO. 3), or a formylated derivative thereof.


SEQUENCE LISTING (17 sequences)

SEQ ID NO: 1 - protein, 380 aa, Homo sapiens WNT5A
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 2 - protein, 6 aa, Homo sapiens
  MOD_RES 1..1: Xaa in position 1 is methionine or nor-leucin
  MOD_RES 4..4: Xaa in position 4 is cysteine or alanine
XDGXEL

SEQ ID NO: 3 - protein, 6 aa, Homo sapiens
MDGCEL

SEQ ID NO: 4 - protein, 7 aa, Homo sapiens
GMDGCEL

SEQ ID NO: 5 - protein, 8 aa, Homo sapiens
EGMDGCEL

SEQ ID NO: 6 - protein, 9 aa, Homo sapiens
SEGMDGCEL

SEQ ID NO: 7 - protein, 10 aa, Homo sapiens
TSEGMDGCEL

SEQ ID NO: 8 - protein, 11 aa, Homo sapiens
KTSEGMDGCEL

SEQ ID NO: 9 - protein, 12 aa, Homo sapiens
NKTSEGMDGCEL

SEQ ID NO: 10 - protein, 13 aa, Homo sapiens
CNKTSEGMDGCEL

SEQ ID NO: 11 - protein, 14 aa, Homo sapiens
LCNKTSEGMDGCEL

SEQ ID NO: 12 - protein, 15 aa, Homo sapiens
RLCNKTSEGMDGCEL

SEQ ID NO: 13 - protein, 16 aa, Homo sapiens
GRLCNKTSEGMDGCEL

SEQ ID NO: 14 - protein, 17 aa, Homo sapiens
QGRLCNKTSEGMDGCEL

SEQ ID NO: 15 - protein, 18 aa, Homo sapiens
TQGRLCNKTSEGMDGCEL

SEQ ID NO: 16 - protein, 19 aa, Homo sapiens
GTQGRLCNKTSEGMDGCEL

SEQ ID NO: 17 - protein, 20 aa, Homo sapiens
LGTQGRLCNKTSEGMDGCEL